(19) United States
(12) Patent Application Publication
(10) Pub. No.: US 2021/0361545 A1
(43) Pub. Date: Nov. 25, 2021

(54) COSMETIC COMPOSITION FOR IMMEDIATE WRINKLE IMPROVEMENT AND ELASTICITY ENHANCEMENT

(71) Applicant: LG HOUSEHOLD & HEALTH CARE LTD., Seoul (KR)

(72) Inventors: No-Jin PARK, Daejeon (KR); Hyeong-Jin CHO, Daejeon (KR); Nae-Gyu KANG, Daejeon (KR); Ki-Young KIM, Daejeon (KR)

(73) Assignee: LG HOUSEHOLD & HEALTH CARE LTD., Seoul (KR)

(21) Appl. No.: 16/768,459

(22) PCT Filed: Jul. 6, 2018
(86) PCT No.: PCT/KR2018/007713

§ 371 Date: May 29, 2020

(30) Foreign Application Priority Data
Dec. 1, 2017 (KR) 10-2017-0164288

(51) Int. Cl. A61K 8/25; A61K 8/02; A61K 8/81; A61K 8/73; A61Q 19/08

(52) U.S. Cl. CPC A61K 8/25; A61K 8/0241; A61K 8/8158; A61K 8/731; A61K 2800/5426; A61K 2800/60; A61K 2800/591; A61K 2800/805; A61Q 19/08

(57) ABSTRACT

The present invention relates to a cosmetic composition for immediately improving skin conditions such as skin wrinkles, skin elasticity, and the like, and specifically, it comprises an anionic particle and a polymer which ionically interacts with the anionic particle as active ingredients, and the composition shows wrinkle improvement and elasticity enhancement effects immediately without skin irritation after applied to skin, and excellently exhibits a wrinkle improvement effect in a cosmetic composition of weakly acidic or neutral pH and has outstanding formulation stability.

### TECHNICAL FIELD

The present application claims the benefit of priority based on Korean Patent Application No. 10-2017-0164288 filed on Dec. 1, 2017, and the entire contents disclosed in the description and drawings of the corresponding allocation are incorporated in the present application.

The present invention relates to a cosmetic composition for immediately improving skin conditions such as skin wrinkles, skin elasticity, and the like.

### BACKGROUND ART

Most of cosmetics for wrinkle improvement that are currently on sale improve wrinkles by the principle of preventing decomposition and promoting synthesis of skin components such as collagen, elastin, hyaluronic acid, and the like, as the active ingredient penetrates the skin. However, this approach has a disadvantage in that there is a difference in the effect depending on the skin absorption rate of the ingredient having a wrinkle improvement effect, and it takes a long time for consumers to experience the effect.

Therefore, products, which consumers can experience the wrinkle improvement effect as soon as they apply them, are being released. This method is largely divided into two ways. The first is a method for covering wrinkles using a particle that can cause diffused reflection. The second is a method for improving wrinkles using a material that causes shrinkage when applied to skin, and products are manufactured using water soluble polymers or hydrophilic polyhydric silicates. However, these two methods have problems in that the persistence is weakened by moisture after application, and in particular, due to agglomeration between particles, whitening and cracking occur, thereby rather making skin defects prominent. In addition, when the hydrophilic polyhydric silicates are applied to a formulation, problems have been found in that the effect is significantly reduced in the acidity (pH 3˜9) applicable to cosmetics.

In order to solve such a high pH, it has been reported that chitosan is applied to bentonite (Korean Patent Publication No. 10-2016-0002896), but the reality is that it is difficult to use chitosan due to safety issues related to allergies.

Accordingly, there is a need to develop a cosmetic composition which has no irritation to skin and can improve wrinkles.

### DISCLOSURE

### Technical Problem

The present invention is to provide a cosmetic composition having an effect of improving skin wrinkles or skin elasticity immediately.

### Technical Solution

To achieve the aforementioned object, the present invention provides a cosmetic composition for immediate wrinkle improvement and elasticity enhancement comprising an anionic particle and a polymer which ionically interacts with the anionic particle as active ingredients.

The anionic particle of the cosmetic composition according to the present invention is characterized by being at least one selected from the group consisting of tin oxide, titanium dioxide, silicon dioxide, manganese dioxide, magnetite, cobalt oxide, cerium oxide, alumina, zinc oxide, silica, clay minerals and synthetic clay and silicate compounds.

The polymer which ionically interacts with the anionic particle of the cosmetic composition according to the present invention is characterized by being at least one selected from the group consisting of polyquaternium-based polymers, silicone quaternium-based polymers, sodium polyquaternium crosspolymers, polylysine, acrylamide-based copolymers, cationic cellulose polymers, poly(dimethyldiallyl ammonium chloride, quaternary ammonium by reaction of dimethylsulfate and vinylpyrrolidone/dimethylaminoethyl methacrylate copolymers, quaternary ammonium from methylvinylimidazolium chloride and vinylpyrrolidone, polymers from trimethyl ammonium ethyl methacrylate chloride, vinylpyrrolidone-based polymers, and dimethylpolysiloxane terminally substituted with a quaternary ammonium group.

The anionic particle of the cosmetic composition according to the present invention is characterized by being comprised in an amount of 0.01 to 20% by weight (w/w) based on the total composition.

The polymer which ionically interacts with the anionic particle of the cosmetic composition according to the present invention is characterized by being comprised in an amount of 0.01 to 15% by weight (w/w) based on the total composition.

The anionic particle of the cosmetic composition according to the present invention is characterized by being comprised in an amount of 0.1 to 5 times of the weight of the polymer which ionically interacts with the anionic particle.

The cosmetic composition according to the present invention is characterized by being pH 3 or more or 9 or less.

### Advantageous Effects

The present invention shows wrinkle improvement and elasticity enhancement effects immediately without skin irritation after applied to skin, and excellently exhibits a wrinkle improvement effect in a cosmetic composition of weakly acidic or neutral pH, and has outstanding formulation stability.

### BRIEF DESCRIPTION OF THE DRAWINGS

FIG. 1 is a graph of the result of shrinkage evaluation of the composition according to the present invention.

FIG. 2 is a photograph of evaluating the formulation stability of the composition according to the present invention.

FIG. 3 is a photograph of confirming the immediate skin wrinkle improvement effect of the composition of the present invention.

### MODE FOR INVENTION

To achieve the aforementioned object, the present invention is characterized by a cosmetic composition for immediate wrinkle improvement and elasticity enhancement comprising an anionic particle and a polymer which ionically interacts with the anionic particle as active ingredients.

The anionic particle of the cosmetic composition according to the present invention is a particle which shows a zeta-potential value less than 0 in the final composition pH and is available for cosmetics, and may be tin oxide (SnO₂), titanium dioxide (TiO₂), silicon dioxide (SiO₂), manganese dioxide (MnO₂), magnetite (Fe₃O₄), cobalt oxide (Co₃O₄), cerium oxide (CeO₂), alumina (Al₂O₃), zinc oxide (ZnO), silica, clay minerals and synthetic clay and silicate compounds, but not limited thereto.

The clay mineral and synthetic clay which can be used in the present invention may be mica, zeolite, laponite, bentonite, saponite, montmorillonite, hectorite, beidilite, sillimanite, wollastonite, and the like, but not limited thereto.

The silicate compound which can be used in the present invention may be aluminum calcium magnesium potassium sodium zinc silicate, aluminum calcium sodium silicate, aluminum iron calcium magnesium germanium silicate, aluminum iron calcium magnesium zirconium silicate, aluminum iron silicate, aluminum silicate, ammonium fluorosilicate, ammonium silver zinc aluminum silicate, calcium aluminum borosilicate, calcium magnesium silicate, calcium silicate, calcium sodium borosilicate, calcium sodium phosphosilicate, calcium titanium borosilicate, C24-28 alkyldimethylsiloxy trimethylsiloxysilicate, cetyldimethylsiloxy trimethylsiloxysilicate, diisostearoyl trimethylolpropane siloxy silicate, dilauroyl trimethylolpropane siloxy silicate, dimethicone/vinyltrimethylsiloxysilicate crosspolymer, dimethiconol/caprylylsilsesquioxane/silicate crosspolymer, dimethiconol/methylsilanol/silicate crosspolymer, dimethiconol/propylsilsesquioxane/silicate crosspolymer, dimethiconyl silicate, ethyl polysilicate, hydroxypropyltrimonium hydrolyzed rice protein/siloxysilicate, hydroxypropyltrimonium hydrolyzed wheat protein/siloxysilicate, laurdimonium hydroxypropyl hydrolyzed wheat protein/siloxysilicate, lithium magnesium silicate, lithium magnesium sodium silicate, magnesium aluminometasilicate, magnesium aluminum silicate, magnesium fluorosilicate, magnesium potassium fluorosilicate, magnesium silicate, magnesium sodium fluorosilicate, magnesium trisilicate, methoxycinnamoylpropyl silsesquioxane silicate, methylsilanol/silicate crosspolymer, phenylpropyldimethylsiloxysilicate, polymethyl/phenylsilsesquioxane silicate, polymethylsilsesquioxane/trimethylsiloxysilicate, polyphenylsilsesquioxane/trimethylsiloxysilicate, potassium fluorosilicate, potassium silicate, silver borosilicate, sodium fluorosilicate, sodium magnesium aluminum silicate, sodium magnesium fluorosilicate, sodium magnesium silicate, sodium metasilicate, sodium potassium aluminum silicate, sodium silicate, sodium silver aluminum silicate, tetraethyl orthosilicate, tetra geranyl/methyldecenyl silicate, trifluoropropyldimethyl/trimethylsiloxysilicate, trimethylsiloxysilicate, trimethylsiloxysilicate/dimethicone crosspolymer, trimethylsiloxysilicate/dimethiconol crosspolymer, tromethamine magnesium aluminum silicate, vinyldimethyl/trimethylsiloxysilicate/dimethicone crosspolymer, vinyldimethyl/trimethylsiloxysilicate stearyl dimethicone crosspolymer, zinc borosilicate, zinc silicate, sodium silicate, aluminum silicate, zirconium silicate, and the like, but not limited thereto.

Specifically, it may be at least one selected from the group consisting of titanium dioxide, alumina, zinc oxide, silica, mica, laponite, bentonite, saponite, montmorillonite, hectorite, beidilite, sodium silicate and aluminum magnesium silicate, and more specifically, it may be at least one selected from the group consisting of silica, laponite and bentonite.

The polymer of the cosmetic composition according to the present invention is a polymer which can ionically interacts with the aforementioned anionic particle, and is a cationic polymer of which whole molecule is positively charged or an active polymer which has a cationic residue in the molecule and can provide shrinkage to skin through interaction with the anion. Therefore, it may comprise a polymer which is cationic by receiving a hydrogen ion from an anionic particle or has a cationic group in the cationic final composition pH.

The polymer of the present invention may be, but not limited thereto, a cationic polymer and a polyacrylamide-based polymer, and as one example, it may be polyquaternium-based polymers, silicone quaternium-based polymers, sodium polyquaternium crosspolymers, polylysine, acrylamide-based copolymers, cationic cellulose polymers, poly(dimethyldiallyl ammonium chloride, quaternary ammonium by reaction of dimethylsulfate and vinylpyrrolidone/dimethylaminoethyl methacrylate copolymers (for example, vinylpyrrolidone/dimethylaminoethyl methacrylate methosulfate copolymers), quaternary ammonium from methylvinylimidazolium chloride and vinylpyrrolidone, polymers from trimethyl ammonium ethyl methacrylate chloride, vinylpyrrolidone-based polymers, and dimethylpolysiloxane terminally substituted with a quaternary ammonium group.

The polyquaternium-based polymer which can be used in the present invention may be polyquaternium-4, polyquaternium-6, polyquaternium-7, polyquaternium-22, polyquaternium-10, polyquaternium-11 polyquaternium-15, polyquaternium-16, polyquaternium-24, polyquaternium-28, polyquaternium-32, polyquaternium-33, polyquaternium-35, polyquaternium-37, polyquaternium-39, polyquaternium-44, polyquaternium-51, polyquaternium-55, polyquaternium-56, polyquaternium-57, polyquaternium-67, polyquaternium-68, polyquaternium-69, polyquaternium-70, polyquaternium-71, polyquaternium-72, polyquaternium-73, polyquaternium-74, polyquaternium-75, polyquaternium-76, polyquaternium-83, polyquaternium-84, polyquaternium-85, polyquaternium-86, polyquaternium-87, polyquaternium-88, polyquaternium-89, polyquaternium-91, polyquaternium-98, and the like, but not limited thereto.

The silicone quaternium-based polymer which can be used in the present invention may be, but not limited thereto, silicone quaternium-1, silicone quaternium-3, silicone quaternium-8, silicone quaternium-15, silicone quaternium-16, silicone quaternium-18, silicone quaternium-20, silicone quaternium-22, and the like.

The acrylamide-based polymer which can be used in the present invention may be, but not limited thereto, polyacrylamide, acrylamide/ammonium acrylate copolymer, acrylamide/ethalkonium chloride acrylate copolymer, acrylamide/ethyltrimonium chloride acrylate/ethalkonium chloride acrylate copolymer, acrylamides copolymer, acrylamides/DMAPA acrylates/methoxy PEG methacrylate copolymer), acrylamide/sodium acrylate copolymer, acrylamide/sodium acryloyldimethyltaurate/acrylic acid copolymer, acrylamide/sodium acryloyldimethyltaurate copolymer, acrylamidopropyltrimonium chloride/acrylamide copolymer, acrylamide/dimethyldiallyl ammonium chloride copolymer, dimethyldiallyl ammonium chloride/sodium acrylate/acrylamide copolymer, and the like.

The cationic cellulose polymer which can be used in the present invention may be, but not limited thereto, a cationic cellulose derivative from hydroxyethyl cellulose/diallyldimethyl ammonium chloride, a cationic cellulose derivative from hydroxyethyl cellulose/trimethylammonium-substituted epoxide, and the like.

The vinylpyrrolidone-based polymer which can be used in the present invention may be, but not limited thereto, vinylpyrrolidone/dimethylaminopropyl methacrylamide/methacryloylamino propyl lauryl dimethyl ammonium chloride copolymer, vinylpyrrolidone/dimethylaminoethyl methacrylate/vinylcaprolactam copolymer, vinylpyrrolidone/methacrylamidopropyltrimethylammonium chloride copolymer, vinylpyrrolidone/dimethylaminoethyl methacrylate copolymer, vinylpyrrolidone/vinylcaprolactam/dimethylaminopropylacrylamide copolymer, and the like.

Specifically, the polymer which ionically interacts with the anionic particle of the present invention may be at least one selected from the group consisting of polyquaterniumObased polymers, polylysine and acrylamide-based polymers, and more specifically, may be at least one selected from the group consisting of polyquaternium-4, polyquaternium-6, polyquaternium-7, polyquaternium-22, polyquaternium-10, polyquaternium-11 polyquaternium-15, polyquaternium-16, polyquaternium-24, polyquaternium-28, polyquaternium-32, polyquaternium-33, polyquaternium-35, polyquaternium-37, polyquaternium-39, polyquaternium-44, polyquaternium-51, polyquaternium-55, polyquaternium-56, polyquaternium-57, polyquaternium-67, polyquaternium-68, polyquaternium-69, polyquaternium-70, polyquaternium-71, polyquaternium-72, polyquaternium-73, polyquaternium-74, polyquaternium-75, polyquaternium-76, polyquaternium-83, polyquaternium-84, polyquaternium-85, polyquaternium-86, polyquaternium-87, polyquaternium-88, polyquaternium-89, polyquaternium-91, polyquaternium-98, polylysine, polyacrylamide, acrylamide/ammonium acrylate copolymers, acrylamide/ethalkonium chloride acrylate copolymers, acrylamide/ethyltrimonium chloride acrylate/ethalkonium chloride acrylate copolymers, acrylamides copolymers, acrylamides/DMAPA acrylates/methoxy PEG methacrylate copolymers, acrylamide/sodium acrylate copolymers, acrylamide/sodium acryloyldimethyltaurate/acrylic acid copolymers, acrylamide/sodium acryloyldimethyltaurate copolymers, acrylamidopropyltrimonium chloride/acrylamide copolymers, acrylamide/dimethyldiallyl ammonium chloride copolymers, and dimethyldiallyl ammonium chloride/sodium acrylate/acrylamide copolymers, and most specifically, may be polyquaternium-10, polyquaternium-51, polyquaternium-67, polylysine and polyacrylamide.

The anionic particle of the cosmetic composition according to the present invention may be comprised in an amount of 0.01 to 20% by weight (w/w) based on the total composition. More specifically, it may be 0.1 to 15% by weight (w/w), and much more specifically, it may be 1 to 7% by weight (w/w). When it is less than 0.01% by weight, the wrinkle improvement effect is substantially incomplete, and when it is over 20% by weight, the wrinkle improvement effect is inefficient compared to the content increase.

The polymer which ionically interacts with the anionic particle of the cosmetic composition according to the present invention may be comprised in an amount of 0.01 to 15% by weight (w/w) based on the total composition. More specifically, it may be 0.1 to 10% by weight (w/w), and much more specifically, it may be 1 to 5% by weight (w/w). When it is less than 0.01% by weight, the wrinkle improvement effect is substantially incomplete, and when it is over 15% by weight, the wrinkle improvement effect is inefficient compared to the content increase.

The anionic particle of the cosmetic composition according to the present invention may be comprised in an amount of 0.01 to 5 times of the weight of the polymer which ionically interacts with the anionic particle. More specifically, it may be 0.1 to 4 times, and much more specifically, it may be 0.3 to 3 times. When it is less than 0.01 time or over 5 times, as the ratio of the anionic particle and the polymer which ionically interacts with it is not appropriate, it may be difficult to form an effective ionic bond between them.

The cosmetic composition according to the present invention may be pH 3 or more to 9 or less. Specifically, the pH may be 4 or more to 8 or less.

In particular, the cosmetic composition comprising sodium silicate, aluminum magnesium silicate, and the like that are anionic particles may show high pH to exhibit the wrinkle improvement effect, and when the pH is reduced using an excipient for compensate for this, there is a disadvantage in that the wrinkle improvement effect is halved or the stability of the formulation is lowered. However, the present invention has an advantage in that a composition may be stably manufactured by interaction between an anionic particle and a polymer which ionically interacts with it, even if a weakly acidic or neutral (pH 4˜7) composition is manufactured by applying an excipient, and simultaneously, the immediate wrinkle improvement effect is exhibited.

The cosmetic composition according to the present invention exhibits a shrinkage effect in a short time after skin application, thereby showing a skin wrinkle improvement effect within about 5 minutes while being dried after skin application.

The cosmetic composition according to the present invention may be applied to all of common formulations of the cosmetic composition without a particular limitation, and therefore, it may be applied to all of cream, essence, lotion, skin, stick and emulsion in a W/O, O/W or W/O/W type including spray with low viscosity, mist formulation, and the like.

The cosmetic composition according to the present invention may comprise components commonly used for the cosmetic composition, and as one example, it may comprise a common preservative such as a moisturizer, an ultraviolet absorber, an ultraviolet scattering agent, vitamins, plant extracts, a skin astringent, an anti-inflammatory agent, a cell activator, a vasodilator, a blood flow stimulant and a skin function agonist, and a carrier, and the like, in addition to various kinds of additives such as a surfactant, a pH adjusting agent, a coloring matter, a flavoring agent, a pigment, a preservative, a disinfectant, a thickener, an anti-oxidant, a stabilizer, a solubilizer, a sequestering agent, a freshener, a deodorant, an emulsifier, a solubilizing agent, and the like, and its formulation and additive components are not limited to the above contents. In addition, the components may be introduced in an amount commonly used in the skin science field.

The formulation of the cosmetic composition according to the present invention is not particularly limited, but it may comprise cosmetic composition combination components commonly used in the technical field to which the invention pertains, depending on the formulation to be manufactured. The cosmetic composition of the present invention may be formulated into for example, liquid products, lotion, essence, cream, sun lotion, sun cream, makeup base, foundation, BB cream, stick products or balm type products, or the like, but not limited thereto. In addition, depending on the formulation to be manufacture, it may further select and add in combination oils, water, a surfactant, a moisturizer, a lower alcohol, a thickener, a chelating agent, a preservative, a flavoring agent, and the like. Furthermore, the cosmetic composition according to the present invention may comprise a sunscreen, a light scattering agent, and the like, and its formulation and additive components are not limited to the above contents. Moreover, the components may be introduced in an amount commonly used in the skin science field.

The cosmetic composition according to the present invention may comprise a wrinkle improvement cosmetic, a cosmetic for whitening, a sunscreen, a light scattering agent, and the like, to add or enhance the functionality, but not limited thereto. The cosmetic composition according to the present invention may be used by directly applying or spraying to skin depending on its type.

The amount of use and the number of uses per day of the composition is appropriately set according to the user's age, gender, use, severity of symptoms, and the like, but for example, an appropriate amount of the composition may be applied to skin at a frequency of 1 time/day to 5 or 6 times/day.

Hereinafter, the present invention will be described in more detail by examples. However, these are intended to illustrate the present invention in more detail, but the scope of the present invention is not limited thereto.

### [Experimental Example 1] Shrinkage Evaluation

In the present experimental example, to find an appropriate combination ratio of the cosmetic composition for immediate wrinkle improvement of the present invention, after mixing raw materials using water as a solvent, the shrinkage was evaluated using the following method.

(1) Measurement Method

On a thin film silicone surface with a thickness of 0.1 mm and width 4 cm×height 20 cm, the mixed raw materials were applied in a thickness of 100 μm. Then, after drying in a thermo-hygrostat chamber with the humidity of 50% and the temperature of 37° C. conditions, the shrinkage was calculated using the following equation.

$$Shrinkage ( \% ) = \frac{( width of the initial silicone sheet - width of the silicone sheet after drying for 30 minutes )}{( width of the initial silicone sheet )} \times 100 \qquad [ Equation ]$$

(2) Shrinkage According to Kinds of the Anionic Particle

To evaluate the wrinkle improvement efficiency, the shrinkage was measured by mixing polyquaternium-10 to various anionic particles. For comparative evaluation, the shrinkage in case of using the anionic particle only was also measured. The result was shown in the following table.

TABLE 1

| | | | | Laponite (3%) + | Bentonite (3%) + | Silica (3%) + |
|---|---|---|---|---|---|---|
| | Laponite | Bentonite | Silica | cationic polymer | cationic polymer | cationic polymer |
| | (3%) | (3%) | (3%) | (1%) | (1%) | (1%) |
| Shrinkage | 0.50 | 0.75 | 0.50 | 1.50 | 1.00 | 1.25 |

As the result of the experiment, it was confirmed that the anionic particle also showed the skin shrinkage effect, but the effect was more increased in case of mixing it and the ionically interacting polymer, and in particular, laponite had a full 3 times increased skin shrinkage effect when mixed with the ionically interacting polymer than used alone.

(3) Shrinkage According to Kinds of the Ionically Interacting Polymer

To evaluate the wrinkle improvement efficiency, the shrinkage was measured by mixing laponite which is an anionic particle with the excellent effect to various polymers which ionically interacts with the anionic particle. For comparative evaluation, the shrinkage in case of using the polymer alone was also measured. The result was shown in the following table.

TABLE 2

| | Polyacrylamide | polyquaternium- | polyquaternium- | polyquaternium- | Polylysine |
|---|---|---|---|---|---|
| | (1%) | 10(1%) | 51(1%) | 67(1%) | (1%) |
| Shrinkage | 0 | 0 | 0 | 0 | 0 |

TABLE 3

| | Anionic particle | Anionic particle | Anionic particle | Anionic particle | Anionic particle |
|---|---|---|---|---|---|
| | (3%) + polyacrylamide | (3%) + polyquaternium-10 | (3%) + polyquaternium-51 | (3%) + polyquaternium-67 | (3%) + polylysine |
| | (1%) | (1%) | (1%) | (1%) | (1%) |
| Shrinkage | 1.00 | 1.50 | 1.00 | 0.75 | 1.25 |

As the result of the measurement, it was confirmed that there was no skin shrinkage effect when the polymer which ionically interacts with the anionic particle was comprised in an amount of 1% by weight, but the excellent skin shrinkage effect was shown when mixed with the anionic particle.

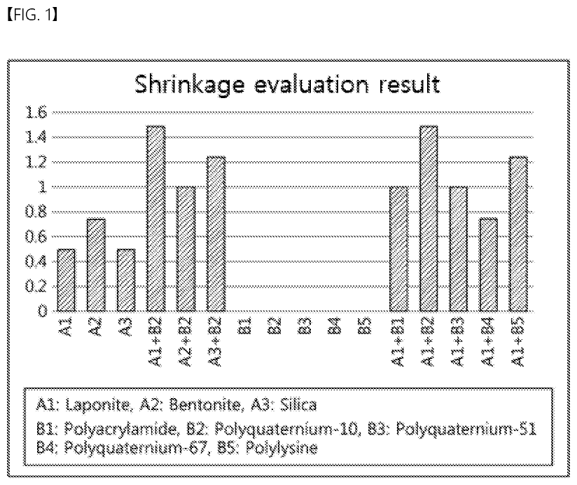

When the result is represented to a graph in combination, it is as FIG. 1. Therefore, it could be confirmed that the cosmetic composition of the present invention more increased the skin shrinkage effect by the anionic particle through mixing with the polymer ionically interacting with it.

### [Experimental Example 2] Formulation Stability Evaluation

In the present experimental example, whether the formulation stability is maintained even in pH 6 due to interaction of the anionic particle and the polymer used in the composition of the present invention was evaluated.

The pH of the control group was adjusted to 6 using a small amount of hydrous citric acid after adding bentonite 3% by weight to purified water 97%. The pH of the experimental group was adjusted to 6 using a small amount of hydrous citric acid after adding polyquaternium-10 1% by weight to purified water 96%. The manufactured formulation was under a stability test using Freeze Thaw method (F/T). After freezing at −20° C. for 24 hours and then raising the temperature at a room temperature, the stability evaluation for elapsed time change was progressed. A total of 5 repeated experiments were conducted, and the result was shown in the following table.

TABLE 4

| Number | 1 time | 2 times | 3 times | 4 times | 5 times |
|---|---|---|---|---|---|
| Control group | Water release | — | — | — | — |
| | occurrence | | | | |
| Experimental | Stable | Stable | Stable | Stable | Stable |
| group | | | | | |

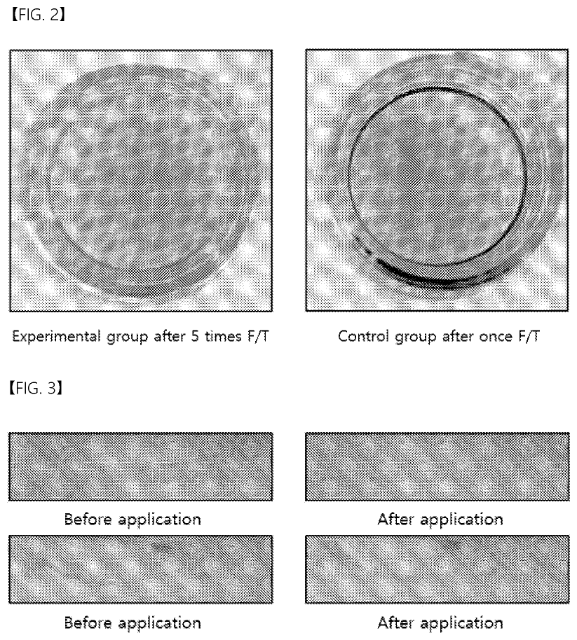

On the other hand, the photograph of the control group in which freeze/thaw (F/T) was conducted once and the photograph of the experimental group after conducting 5 times, according to the above experimental method were included in FIG. 2.

### [Example 1] Manufacture of the Cosmetic Composition

The cosmetic composition exhibiting the immediate skin wrinkle improvement effect of the present invention was manufactured using the raw materials shown in the following table.

TABLE 5

| Number | INCI Name | #50 |
|---|---|---|
| 1 | Sodium magnesium fluorosilicate | 1 |
| 2 | Polymethylsilsesquioxane | 1 |
| 3 | Silica | 1.25 |
| 4 | Citric acid | 0.15 |
| 5 | Purified water 1 | 40 |
| 6 | Polyquaternium-51 | 5 |
| 7 | PANTHENOL | 2 |
| 8 | Glycerin | 1 |
| 9 | 1,2-Hexanediol | 1 |
| 10 | MYRISTICA FRAGRANS (NUTMEG) EXTRACT | 3 |
| 11 | Polyacrylamide | 1.5 |
| 12 | Purified water 2 | 43.1 |
| | Total | 100 |

The raw materials of numbers 1˜3 were added to the raw material of number 5, and they were mixed for 30 minutes while raising the temperature from 40° C. Then, adding the raw material of number 4, they were mixed for 10 minutes. Meanwhile, the raw materials of numbers 6˜11 were added to the raw material of number 12 and then they were mixed for 30 minutes while heating to 40° C. Then, the mixed solution of numbers 1˜5 prepared in advance was added, and they were mixed for 15 minutes, thereby manufacturing the cosmetic composition.

For the manufactured cosmetic composition, the shrinkage was evaluated using the shrinkage measurement method used in the experimental example 1. As the result of evaluation, 0.75% shrinkage was shown. On the other hand, as the result of evaluation using a pH meter, the manufactured cosmetic composition exhibited weak acidity.

### [Example 2] Confirmation of the Immediate Skin Wrinkle Improvement Effect

In the present experimental example, the immediate skin wrinkle improvement effect of the cosmetic composition exhibiting the immediate skin wrinkle improvement effect of the present invention manufactured in the example 1 was to be confirmed. Before the experiment, eyes of two experimenters were photographed, and in 5 minutes after applying the cosmetic composition of the present invention, the eyes of the experiments were photographed, to confirm the wrinkle and eyebag improvement effect utilizing naked eyes and a program (used equipment: Vsiosphere 3D, Primos).

As the result of the confirmation, as could be confirmed in FIG. 3, it could be confirmed that eye wrinkles of the 2 experimenters were immediately improved. In addition, the cosmetic composition of the present invention was weakly acidic and had no irritation to skin, and was a cream formulation and had a good feeling of use. Furthermore, it exhibited a pH between weak acidity to neutrality and had no irritation to skin, and could freely create facial expressions with moderate pulling and did not display even when applied to skin, and therefore its feeling of use was very excellent.

Moreover, as the result of calculating by the program, the wrinkles were improved by 14.76% compared to those before application, and the eyebags were reduced by 37.05%.

In the above, the applicant has described preferable examples of the present invention, but these examples are only examples that implement the technical spirit of the present invention, and it should be interpreted that any modification or alteration belongs to the scope of the present invention as long as the technical spirit of the present invention is implemented.

1. A method of immediately improving wrinkle and enhancing elasticity, comprising:
applying a cosmetic composition comprising an anionic particle and a polymer which ionically interacts with the anionic particle to a subject in need thereof.

2. The method according to claim 1, wherein the anionic particle is at least one selected from the group consisting of tin oxide (SnO₂), titanium dioxide (TiO₂), silicon dioxide (SiO₂), manganese dioxide (MnO₂), magnetite (Fe₃O₄), cobalt oxide (Co₃O₄), cerium oxide (CeO₂), alumina (Al₂O₃), zinc oxide (ZnO), silica, clay minerals and synthetic clay and silicate compounds.

3. The method according to claim 1, wherein the polymer which ionically interacts is at least one selected from the group consisting of polyquaternium-based polymers, silicone quaternium-based polymers, sodium polyquaternium crosspolymers, polylysine, acrylamide-based copolymers, cationic cellulose polymers, poly(dimethyldiallyl ammonium chloride, quaternary ammonium by reaction of dimethylsulfate and vinylpyrrolidone/dimethylaminoethyl methacrylate copolymers, quaternary ammonium from methylvinylimidazolium chloride and vinylpyrrolidone, polymers from trimethyl ammonium ethyl methacrylate chloride, vinylpyrrolidone-based polymers, and dimethylpolysiloxane terminally substituted with a quaternary ammonium group.

4. The method according to claim 1, wherein the anionic particle is at least one selected from the group consisting of titanium dioxide, alumina, zinc oxide, silica, mica, laponite, bentonite, saponite, montmorillonite, hectorite, beidilite, sodium silicate and aluminum magnesium silicate, and
the polymer which ionically interacts is at least one selected from the group consisting of polyquaternium-4, polyquaternium-6, polyquaternium-7, polyquaternium-22, polyquaternium-10, polyquaternium-11 polyquaternium-15, polyquaternium-16, polyquaternium-24, polyquaternium-28, polyquaternium-32, polyquaternium-33, polyquaternium-35, polyquaternium-37, polyquaternium-39, polyquaternium-44, polyquaternium-51, polyquaternium-55, polyquaternium-56, polyquaternium-67, polyquaternium-68, polyquaternium-69, polyquaternium-70, polyquaternium-71, polyquaternium-72, polyquaternium-73, polyquaternium-74, polyquaternium-75, polyquaternium-76, polyquaternium-83, polyquaternium-84, polyquaternium-85, polyquaternium-86, polyquaternium-87, polyquaternium-88, polyquaternium-89, polyquaternium-91, polyquaternium-98, polylysine, polyacrylamide, acrylamide/ammonium acrylate copolymers, acrylamide/ethalkonium chloride acrylate copolymers, acrylamide/ethyltrimonium chloride acrylate/ethalkonium chloride acrylate copolymers, acrylamides copolymers, acrylamides/DMAPA acrylates/methoxy PEG methacrylate copolymers, acrylamide/sodium acrylate copolymers, acrylamide/sodium acryloyldimethyltaurate/acrylic acid copolymers, acrylamide/sodium acryloyldimethyltaurate copolymers, acrylamidopropyltrimonium chloride/acrylamide copolymers, acrylamide/dimethyldiallyl ammonium chloride copolymers, and dimethyldiallyl ammonium chloride/sodium acrylate/acrylamide copolymers.

5. The method according to claim 1, wherein the anionic particle is comprised in an amount of 0.01 to 20% by weight (w/w) based on the total composition.

6. The method according to claim 1, wherein the polymer which ionically interacts is comprised in an amount of 0.01 to 15% by weight (w/w) based on the total composition.

7. The method according to claim 1, wherein the anionic particle is comprised in a weight of 0.01 to 5 times of the weight of the polymer which ionically interacts.

8. The method according to claim 1, wherein the cosmetic composition is pH 3 or more to 9 or less.
